SEQUENCE LISTING (4 sequences)

SEQ ID NO: 1 - DNA, 300 bp, Homo sapiens
accaacaaagaagcctccatctgtccatccgtatatctatccaccctaccatccatccattcacccactaattcatccatttattatccatgcatccatctgtccataagtctatccgtccacccaccacttatccatccatccatttacccatcatactcatccattcattcatccagccaccacccatgcactcacctatccacccattcagtcattaatccagtaaaaaattttgagcacctactaccaatcaggccctgcacttggaccttagggtagtgtgtaaataaaacccca

SEQ ID NO: 2 - protein, 93 aa, Homo sapiens
TNKEASICPSVYLSTLPSIHSPTNSSIYYPCIHLSISLSVHPPLIHPSIYPSYSSIHSSSHHPCTHLSTHSVINPVKNFEHLLPIRPCTWTLG

SEQ ID NO: 3 - protein, 8 aa, Artificial Sequence
  Description of Artificial Sequence Illustrative immunogenic peptide
RTNKEASI

SEQ ID NO: 4 - DNA, 23 bp, Artificial Sequence
  modified_base (3): a, t, c or g
acnaayaargargcntcnathtc

(19) United States
(12) Patent Application Publication
(10) Pub. No.: US 2003/0157116 A1
(43) Pub. Date: Aug. 21, 2003

(54) Tlp peptides and dna sequences coding the same

(76) Inventor: Giulio Tarro, Napoli (IT)

Correspondence Address: YOUNG & THOMPSON, 745 SOUTH 23RD STREET 2ND FLOOR, ARLINGTON, VA, 22202

(21) Appl. No.: 10/204,635

(22) PCT Filed: Feb. 20, 2001
(86) PCT No.: PCT/EP01/01857

(51) Int. Cl. A61K 39/00; C07H 21/04; C07K 14/47

(52) U.S. Cl. 424/185.1; 435/320.1; 530/350; 530/388.1; 536/23.5

(57) ABSTRACT

The present invention generally relates to DNA molecules encoding peptides derived from proteins of the TLP complex. More specifically, the invention is directed to a cDNA sequence coding for a protein of the 100 kDa TLP complex.

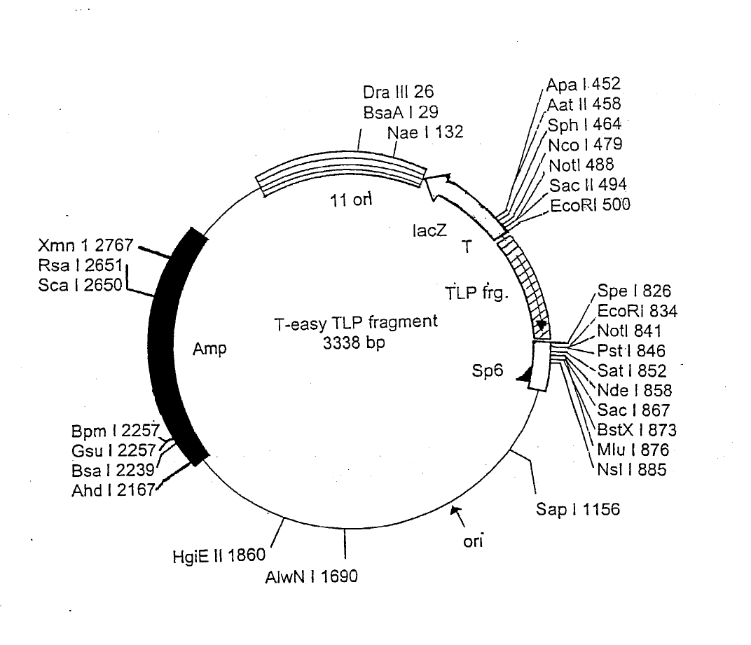

The present invention relates to DNA molecules encoding peptides derived from proteins of the TLP complex. More specifically, the invention is directed to a cDNA sequence coding for a peptide of the 100 kDa TLP complex.

Under the acronym TLP (“Tumor Liberated Particles”), protein complexes present in human tumor cells, particularly in lung carcinoma, are indicated. TLPs were isolated for the first time from tumor tissues following the procedure disclosed in EP283433. Afterwards, certain proteins forming the TLP complexes were identified. A 214 kDa TLP protein prevalently expressed in lung carcinoma is disclosed in Oncology, 1983, 40:248-253.

Epitopes specific for that protein and antibodies against it are disclosed in WO098/01462. In addition, EP649433 discloses a 100 kDa protein isolated from lung carcinoma, as well as immunogenic peptides derived therefrom. Among those, the peptide RTNKEASI was used to produce polyclonal antibodies recognizing the 100 kDa TLP complex. Such antibodies were used for the characterisation of the TLP expression pattern in tissues of different origin and for the validation of the same protein as tumor marker (WO98/15282). The study of TLP expression in different tumor cell lines has revealed similar expression levels among cell lines derived from breast carcinoma (MCF7) and colorectal carcinoma (HT29).

Furthermore, the in vitro treatment with chemotherapeutic agents was shown to increase the expression of TLP antigen in a tumor cell population (NSCLC), whereas the 100 kDa TLP protein, as well as the peptides derived therefrom, lymphocytes with specific antitumor effects (WO98/15282).

It is evident the importance to provide a larger variety of antigenic and immunogenic peptides from the tumor protein TLP complex, as well as an effective means for the extensive production of the same TLP proteins, for diagnostic or therapeutic uses. To that purpose, the inventors have obtained the cDNA sequence encoding a peptide of the 100 kDa TLP complex, as described hereafter.

Lysates of tumor cells expressing TLP were prepared, from which the total RNA was extracted and then subjected to RT-PCR with pairs of oligonucleotide primers having degenerate sequences corresponding to the TNKEASI peptide (forward primer) and, respectively, random hexanucleotide sequences (reverse primer). The amplification product was cloned in a suitable vector and then sequenced. Inside it, an ORF of 300 bases was identified, whose sequence is reported in SEQ ID N. 1. The amino acid sequence encoded by the ORF is reported in SEQ ID N. 2.

According to a first aspect, the invention provides a nucleic acid molecule comprising the sequence SEQ ID N. 1, the variants thereof due to genetic code degeneration, and the nucleic acid molecules hybridising to the complementary strand of SEQ ID N. 1 under stringent conditions.

The invention further comprises an expression vector that contains a nucleic acid molecule herein disclosed. The vector can be a plasmid, cosmid, virus, bacteriophage, or any other vector commonly used in genetic techniques, which, in addition to the coding sequence, may comprise control elements, such as sequences regulating the transcription, including start and stop codons, enhancers, promoters, signal sequences and the like. Furthermore, the invention comprises eukaryotic or prokarvotic cells transfected or transformed with said vector.

The complete sequence of a TLP protein enables in vitro preparations of large quantities of it by genetic engineering. The availability of such antigen preparations allows for deep investigations on the role of TLP in human malignancy. It also enables the preparation of an assay for early diagnosis of the corresponding tumors and the generation of a specific anti cancer vaccine.

A further aspect of the invention relates to a peptide encoded by the DNA sequence of SEQ ID N. 1, as reported in SEQ ID N. 2, or a fragment thereof. Such peptides can be prepared with recombinant DNA procedures, using the described cDNA, or with chemical synthesis, following known procedures (e.g. see “Merrifield, (1986) Science 232:341-347”, or “Barany and Merrifield, 1979, The peptides, Gross and Meienhofer, eds NY Academic Press, 1-284”). The synthesis can be carried out in solution or in solid phase with an automatic synthesizer. One or more amino acid residues can be replaced by different L- or D-residues, so as to maintain the immunogenic effects, or they can be chemically modified, for instance by amidation of the carboxy terminus, by linking lipophilic groups (e.g. myristyl), or by glycosylation or conjugation with other peptides, in order to improve properties like immunogenicity, selectivity of induction of the immune response or bioavailability after administration. The peptides can also be chemically derivatized on their lateral chains, for instance on free carboxy groups, to form salts, esters, hydrazides. Furthermore, said peptides can be conjugated to known epitopes in order to induce a higher cytotoxic or helper immune response against tumors.

The immunogenic activity of the peptides can be easily determined by means of in vitro tests, for instance using t-lymphocyte propagation or proliferation essays (Protti M. P. et al., 1990, J. Immunol. 144:1711-1720), cytotoxicity essays (Protti M. P. et al., 1996, Cancer Res. 56:1210-1213), or binding essays with MHC molecules.

According to a preferred embodiment, the peptides derived from SEQ ID N. 2 are used to generate TLP-specific antibodies. The latter can be monoclonal or polyclonal and recognize the TLP epitopes specific for the peptides herein disclosed. Such antibodies are preferably used in immunoessays to detect TLP expression in tumors.

According to a further aspect, the invention provides the use of the above nucleic acid molecules or peptides for the preparation of a pharmaceutical composition for the preventive or therapeutic treatment of tumors, especially of lung tumors. A typical application involving the use of the cDNA is the preparation of DNA vaccines, which can be effected as taught in U.S. Pat. No. 5,593,972 or U.S. Pat. No. 5,580,859. The peptides can also be used in combination with different chemotherapeutic agents, which are known to increase the effects thereof (WO98/15282).

The compositions in accordance with the invention contain an effective amount of DNA or peptides together with pharmaceutically acceptable excipients. By “effective amount” is meant the amount sufficient to activate lymphocytes and to trigger a cellular or humoral response against tumors. According to a preferred embodiment, the pharmaceutical compositions are used in the preventive vaccination of subjects with susceptibility to neoplasias or in the therapeutic vaccination of neoplastic patients. The term “vaccination” in this context is referred either to the active vaccination, i.e. the in vivo administration of the peptides or cDNA to activate the immune response directly in the patient, or to the passive vaccination, i.e. the use of peptides for the in vitro activation of T cytotoxic lymphocytes and their subsequent re-inoculation in the patient. The procedures for the preparation and use of vaccines are known to anyone skilled in the art; an extensive description is given for example in Paul, Fundamental Immunology, Raven Press, New York (1989) or Cryz, S. J., Immunotherapy and Vaccines, VCH Verlagsgesselschaft (1991). Vaccines are usually prepared in form of solutions, injectables, suspensions, but also in form of solid or liposome-based preparations. The immunogenic ingredients can be mixed with pharmaceutically acceptable excipients, such as emulsifiers, buffering agents, adjuvants, so as to increase the vaccine effectiveness.

The following examples illustrate the invention in greater detail.

### EXAMPLES

### Example 1

### Isolation and Cloning of TLP cDNA

Total RNAs were extracted from several cell lines (HT-29, SAOS-2, A 549, MCF-7, H23, H157, H1819, W138) with RNAzol B reagent (TEL-TEST, INC) and reverse transcribed using the Reverse Transcription System (PROMEGA). Polymerase chain reaction (PCR) was carried out for 35 cycles (1 min at 95° C., 2 min at 40° C. and 1 min at 72° C.) using an upstream degenerate oligonucleotide: ACN AAY AAR GAR GCN TCN ATH TC, that corresponds to the amino acid sequence RTNKEASI and Random Hexamers as the downstream primer. PCR products were electrophoresed on a 1% agarose gel containing ethidium bromide. The PCT products were cloned in the pGEM-T easy vector (PROMEGA) (Figure). Resulting plasmid clones were sequenced with the chain termination method using the Applied Biosystems model 373A DNA sequencer. An open reading frame has been deducted (SEQ ID N. 1).

### Example 2

### Production and Characterization of Antibodies

Rabbit anti-TLP serum was obtained by immunizing four rabbits subcutaneously with 0.5 mg of the peptide of SEQ ID N. 2. in 0.5 ml of phosphate-buffered saline (PBS), mixed with 0.5 ml of Freund's complete adjuvant. Booster injections with incomplete adjuvant were given at 2-week intervals. Sera were collected on alternate weeks via ear vein bleeds. Titers of antisera were performed by radioimmunoassay on 96-well microtiter plates. Wells were coated with a fixed concentration of peptide antigen, washed, and incubated with various dilutions of sera. The bound immunoglobuline was detected with ¹²⁵I-labeled protein A and quantified by radioimmunoassay. At a dilution of 1:1,000, all sera showed a positive staining reaction with the peptide antigen. Preimmune sera from the corresponding rats were also tested and non significant reactivity was detected.

Western Blot

Tumor cell lysates were prepared by resuspending pelletted cells in 200 μl lysis buffer (50 mMTris, 5 mM EDTA, 250 mM Na Cl, 50 mM NaF, 0.1% Triton, 0.1 mM Na3VO4, plus protease inhibitors). 50 μg of protein extract was run on a 8% polyacrylamide gel. Proteins within the polyacrylamide gel were transferred to a PVDF membrane (Millipore) in CAPS buffer (10 mM CAPS, 20% methanol, pH 11). The membrane was blocked with 5% milk in TBS-T buffer (2 mM Tris, 13.7 mM NaCl, 0.1% Tween-20, pH 7.6) and then washed in TBS-T. Affinity purified anti-TLP antibodies were incubated with the membrane in 3% milk and then washed in TBS-T. The membrane was then incubated with a rabbit anti-rabbit Ab coupled with horseradish peroxidase and washed in TBS-T. The presence of secondary antibody bound to the membrane was detected using the ECL system (Dupont NEN). A specific staining was detected in correspondence of the tumor cell Iysates. Preimmune sera and control lysates did not show a significant-reactivity.

1. A nucleic acid molecule encoding a TLP peptide having the sequence SEQ ID N. 2.

2. A nucleic acid molecule according to claim 1, having the sequence SEQ ID N. 1.

3. The peptide of SEQ ID N. 2.

4. Monoclonal or polyclonal antibodies against the peptide of claim 3.

5. A vector containing the nucleic acid molecules according to claims 1 or 2.

6. A pharmaceutical composition comprising the nucleic acid molecules or the peptide according to claims 1-2 and respectively 3.

7. A composition according to claim 6, in form of vaccine.

8. A composition according to claims 6-7, for use in the prevention or treatment of tumors.

9. A composition according to claim 8, for use in the prevention or treatment of lung tumors.
